(19) United States
(12) Patent Application Publication
(10) Pub. No.: US 2019/0274965 A1
(43) Pub. Date: Sep. 12, 2019

(54) PHARMACEUTICAL COMPOSITION COMPRISING AN ANTIEMETIC AGENT AND METHOD FOR THE PREPARATION THEREOF

(71) Applicant: PHARMATHEN S.A., PALLINI-ATTIKIS (GR)

(72) Inventor: EVANGELOS KARAVAS, PALLINI ATTIKIS (GR)

(73) Assignee: PHARMATHEN S.A., PALLINI-ATTIKIS (GR)

(21) Appl. No.: 16/349,226

(22) PCT Filed: Nov. 15, 2017
(86) PCT No.: PCT/EP2017/025335

§ 371 Date: May 10, 2019

(30) Foreign Application Priority Data
Nov. 17, 2016 (GR) 20160100607

(51) Int. Cl. A61K 9/50; A61K 31/5377

(52) U.S. Cl. CPC A61K 9/5047; A61K 31/5377; A61K 9/5089; A61K 9/5073; A61K 9/5015; A61K 9/5031; A61K 9/5042

(57) ABSTRACT

The present invention relates to a stable pharmaceutical formulation of solid dosage forms for oral administration comprising a therapeutically effective amount of an antiemetic agent in particular Aprepitant and an effective amount of surface stabilizer in order to improve bioavailability. It also relates to a process for the preparation thereof.

### TECHNICAL FIELD OF THE INVENTION

The present invention relates to a stable pharmaceutical formulation for oral administration containing a therapeutically effective quantity of an antiemetic agent such as Aprepitant and a method for the preparation thereof.

### BACKGROUND OF THE INVENTION

Nausea and vomiting (emesis) are devastating side-effects of treatment with antineoplastic agents. The patients usually describe nausea and vomiting as the most feared effects of chemotherapy. Moreover, nausea and vomiting can cause metabolic imbalances, degeneration of self-care and functional ability, nutrient depletion, anorexia, decline in patient's performance and mental status, wound dehiscence and oesophageal tear.

During the last few decades, definite progress has been made in the management of chemotherapy induced nausea and vomiting (CINV). The introduction of antiemetic drugs including dopamine receptor blockers and serotonin receptor antagonists helped to lessen or prevent nausea and vomiting up to certain extent. But the effectiveness of these drugs remained largely limited to acute emesis even when started before the first dose of the anticancer drug at each cycle of treatment.

Drugs which block substance P from binding to neurokinin type 1 (NK₁) receptors can be of use as antiemetic agents as these neurotransmitters are implicated in the pathoetiology of emesis. Aprepitant is the first member of this new class of antiemetic drugs which is a potent, selective, CNS-penetrant, oral, nonpeptide antagonist of NK₁ receptor which inhibits both the acute and delayed emesis induced by cytotoxic chemotherapeutic drugs.

The chemical name of Aprepitant is 5-[[(2R,3S)-2-[(1R)-1-[3,5-bis(trifluromethyl) phenyl]ethoxy]-3-(4-fluorophenyl)-4-morpholinyl]methyl]-1,2-dihydro-3H-1, 2, 4-triazol-3-one. It is an off-white crystalline solid. Its molecular formula is C₂₃H₂₁F₇N₄O₃ corresponding to a molecular weight of 534.427. It has a very limited solubility in water and a reasonably high solubility in non-polar molecules such as oils. This would, therefore, suggest that Aprepitant as a whole, despite having components that are polar, is a non-polar substance.

WO2012/136816 A2 discloses an oral pharmaceutical composition comprising coated particles of a complex of at least one active agent with an ion-exchange resin, wherein said particles are coated with a bioadhesive coating layer comprising at least one bioadhesive material.

EP 2582697 A1 discloses a stable nanostructured Aprepitant composition comprising nanostructured Aprepitant having an average particle size of less than about 200 nm, sodium dodecyl sulfate and Soluplus, wherein the composition is prepared in a continuous flow reactor, preferably in a microfluidic based continuous flow reactor.

Although each of the patents above represents an attempt to overcome the stability and solubility problems associated with pharmaceutical compositions comprising Aprepitant, there still exists a need for a pharmaceutical composition which avoids such problems.

### SUMMARY OF THE INVENTION

It is an object of the present invention to provide a stable solid pharmaceutical composition for oral administration containing an antiemetic agent and in particular Aprepitant as an active ingredient, which overcomes the deficiencies of the prior art.

Another object of the present invention is to provide a pharmaceutical composition in the form of a capsule comprising Aprepitant that overcomes the low water solubility of the active ingredient and has acceptable pharmacotechnical properties.

It is another object of the present invention to provide an oral solid dosage formulation comprising Aprepitant as an active ingredient, which is bioavailable and with sufficient self-life.

A major object of the present invention is the selection of the optimal combination of pharmaceutical acceptable excipients and method of preparation in order to achieve the appropriate dissolution profile and stability for the finished dosage form. Said dosage form affords predictable and reproducible drug release rates in order to achieve better treatment to a patient.

In accordance with the above aspects of the present invention, a pharmaceutical composition for oral administration is provided comprising Aprepitant as an active ingredient and at least one surface stabilizer to improve bioavailability.

According to another embodiment of the present invention, a process for the preparation of a stable, solid dosage form for oral administration, containing Aprepitant as an active ingredient and at least one surface stabilizer to improve bioavailability is provided, which comprises the following steps:
preparing an aqueous suspension of the active ingredient and surface stabilizers;
milling until desirable particle size;
adding the redispersing agent in water;
mixing the solution of redispersing agent with the milled mixture;
spray-coating on solid support;
encapsulating.

Other objects and advantages of the present invention will become apparent to those skilled in the art in view of the following detailed description.

### DETAILED DESCRIPTION OF THE INVENTION

For the purposes of the present invention, a pharmaceutical composition comprising an active ingredient is considered to be “stable” if said ingredient degrades less or more slowly than it does on its own and/or in known pharmaceutical compositions.

The present invention provides oral pharmaceutical compositions of Aprepitant that overcome the disadvantages over the other known pharmaceutical compositions in terms of increased oral bioavailability.

It has been unexpectedly found that the addition of surface stabilizers in certain quantity in solid dosage formulations of Aprepitant substantially improves the bioavailability of the composition.

More particularly, the target is achieved when 2-15% by weight of surface stabilizer and more preferably 4-12% by weight of surface stabilizer is comprised in solid dosage formulations of Aprepitant.

The surface stabilizer physically adheres to the active ingredient but it does not chemically bond to or chemically react with the drug. Such chemical bonding or interaction would be undesirable as it could result in altering the function of the drug. Preferred surface stabilizers include sodium lauryl sulfate (SLS), hydroxypropyl cellulose (HPC), HPC-H, HPC-M, hydroxypropyl methyl cellulose (HPMC), carboxymethyl cellulose sodium, methyl cellulose, polyoxyethylene-polyoxypropylene copolymers, polyoxyethylene sorbitan fatty acid esters.

Preferably SLS, HPC-H, HPC-M, HPMC, polyoxyethylene-polyoxypropylene copolymer, polyoxyethylene sorbitan fatty acid esters are used as surface stabilizers in the present invention.

The effect is intensified when Aprepitant is directly mixed with the surface stabilizer and the mixture is milled to obtain D95 more than one micron.

The mechanical means applied to reduce particle size of mixture of active ingredient with surface stabilizers can be a dispersion mill. Suitable dispersion mills are selected from ball mill, attritor mill, vibratory mill, and media mills such as sand mill or a bead mill.

The pharmaceutical compositions of the present invention may also contain one or more additional formulation excipients such as redispersing agents, provided that they are compatible with the active ingredient of the composition, so that they do not interfere with it in the composition and in order to increase the stability of the drug and the self-life of the pharmaceutical product.

Preferred redispersing agents include sugars such as glucose, mannitol, lactose, dextrose, xylitol or sucrose, especially sucrose. The redispersing agent is present in an amount of 10-50% by weight, more preferably 20-45% by weight.

The solution of redispersing agent is mixed with the milled mixture of active ingredient and surface stabilizers and the mixture obtained is sprayed onto a solid support.

Preferred solid supports include sugar, starch, cellulose, especially microcrystalline cellulose spheres or pellets. The solid support is present in an amount of 5-50% by weight, more preferably 10-30% by weight.

The following examples illustrate the preferred embodiments in accordance with the present invention without limiting the scope or spirit of the invention. Compositions 1 & 2 comprise surface stabilizers selected from HPC, HPMC, SLS, polyoxyethylene-polyoxypropylene copolymer, sucrose as redispersing agent and microcrystalline cellulose as solid support.

### EXAMPLES

TABLE 1
Composition 1 & 2

%

| Composition | 1 | 2 |
|---|---|---|
| Aprepitant | 36.36 | 35.71 |
| Microcrystalline cellulose | 19.25 | 21.09 |
| Hydroxypropyl cellulose | 7.40 | — |
| Hydroxypropyl methyl cellulose | — | 7.35 |
| Sodium lauryl sulphate | 0.63 | — |
| Polyoxyethylene-polyoxypropylene copolymer | — | 0.13 |
| Sucrose | 36.36 | 35.71 |

| | |
|---|---|
| Total | 100 |

The preferred compositions according to the present invention (Composition 1 & 2) are prepared according to the following manufacturing process:
Preparing Mixture A by mixing Aprepitant and surface stabilizers and then milling until desirable particle size;
Preparing Solution B by adding sucrose in water;
Mixing Mixture A and Solution B;
Spray coating the final mixture on microcrystalline pellets;
Encapsulating in capsules.

While the present invention has been described with respect to the particular embodiment, it will be apparent to those skilled in the art that various changes and modifications may be made in the invention without departing from the spirit and scope thereof, as defined in the appended claims.

1. A pharmaceutical composition for oral administration comprising an antiemetic agent such as Aprepitant as the active ingredient and an effective amount of one or more surface stabilizer in order to improve bioavailability.

2. The pharmaceutical composition according to claim 1, wherein the antiemetic agent is Aprepitant.

3. The pharmaceutical composition according to claim 1, wherein the surface stabilizer is one or more selected from sodium lauryl sulfate (SLS), hydroxypropyl cellulose (HPC), HPC-M, HPC-H, hydroxypropyl methyl cellulose (HPMC), carboxymethyl cellulose sodium, methyl cellulose, polyoxyethylene-polyoxypropylene copolymers, polyoxyethylene sorbitan fatty acid esters.

4. The pharmaceutical composition according to claim 1, wherein the surface stabilizer is preferably one or more selected from SLS, HPC, HPC-H, HPC-M, HPMC, polyoxyethylene-polyoxypropylene copolymer, polyoxyethylene sorbitan fatty acid esters.

5. The pharmaceutical composition according to claim 1, comprising one or more surface stabilizer in an amount 2-15% by weight of the composition.

6. The pharmaceutical composition according to claim 1, further comprising a redispersing agent and a solid support.

7. The pharmaceutical composition according to claim 6, wherein the redispersing agent is sucrose and the solid support is microcrystalline cellulose.

8. The pharmaceutical composition according to claim 7, comprising sucrose in an amount of 10-50% by weight of the composition and microcrystalline cellulose in an amount of 5-50% by weight of the composition.

9. A process for the preparation of a stable, solid dosage form for oral administration, containing an antiemetic agent such as Aprepitant as an active ingredient and an effective amount of one or more surface stabilizer to improve bioavailability, which comprises the following steps:
Preparing Mixture A by mixing Aprepitant and surface stabilizer(s) and milling it until desirable particle size;
Preparing Solution B by adding sucrose in water;
Mixing Mixture A and Solution B;
Spray coating the final mixture on microcrystalline pellets;
Encapsulating in capsules.

10. The process according to claim 9, wherein the surface stabilizers are selected from HPC, HPMC, SLS, polyoxyethylene-polyoxypropylene copolymer.
